(19) United States
(12) Patent Application Publication
(10) Pub. No.: US 2008/0214502 A1
(43) Pub. Date: Sep. 4, 2008

(54) Apparatus and Method for Dispersing Insecticide

(76) Inventors: Glenn Smogoleski, Spicer, MN (US); Peder K. Davisson, Minneapolis, MN (US)

Correspondence Address: Mr. Peder K. Davisson, Esq.;Davisson & Associates, PA, 3649 Brunswick Avenue North, Minneapolis, MN, 55422, US

(21) Appl. No.: 11/381,205

(22) Filed: May 2, 2006

(51) Int. Cl. A01N 65/00; A61K 47/00; A01P 7/04; F01N 5/00

(52) U.S. Cl. 514/65; 514/772; 60/319

(57) ABSTRACT

A method includes formulating an insecticide that is miscible with a liquid fuel, adding a selected amount of the insecticide to a liquid fuel, and combusting the liquid fuel and insecticide in an internal combustion engine. An apparatus for distributing an insecticide includes an internal combustion engine further including a combustion chamber for burning a mixture of fuel and insecticide, and an exhaust port for placing the combusted fuel and insecticide into the atmosphere near the internal combustion engine. In another embodiment, an apparatus includes an exhaust system, an insecticide reservoir, and a device for coupling the insecticide reservoir and the exhaust system, the device for coupling the insecticide reservoir including an inlet to the exhaust system.

### BACKGROUND

Although some areas of the world are relatively pest-free, most of the world has a constant problem with insects. People working outdoors many times have jobs that seem to exacerbate problems with insects. For example, people that mow lawns or cut down brush are simultaneously disrupting habitat for insects. The insects which have been driven from their habitat will be numerous and will accelerate any pest problem. In other words, a person walking in a brush filled setting will attract insects that happen to be nearby. However, when one is removing the habitat such as by operating a weed whacking device or mowing down vegetation, more insects will be in the area near the person.

People that do such jobs generally apply large amounts of insect repellant with high concentrations of insect repelling chemicals. For example, some heavy duty insect repellant has concentrations of as much as 40% DEET. Many writers suggest using such formulations sparingly. Generally, the insect repellant has limited effectiveness and the person must “escape” the job at times and take a break. In addition, the insect repellant generally wears off and requires periodic reapplication.

In addition, some use fogging machines to treat an area to cut down or substantially reduce the number of insects in a particular area where they will be working or recreating. Fogging generally is done prior to the activity and so the fog will dissipate as time goes on. The fog dissipates faster in windy conditions as the fog is blown away or, if the fog condenses on vegetation, the fog evaporates faster. One option is to choose to forgo the use of insecticide. In many areas of the world this simply is not an option as some insects carry very serious diseases. Regardless of what is written about various formulations of insecticide it seems that persons working or recreating in an environment that includes insects, must choose either to use an insecticide or risk some discomfort and possibly risk getting a very serious disease. Even if one chooses to use an insecticide, workers or recreators must generally interrupt their activity to reapply an insecticide, move a citronella candle or lantern, fog a new area, or reapply fog to the same area so that they can continue their work or fun in the presence of less pests.

### BRIEF DESCRIPTION OF THE DRAWINGS

The invention is pointed out with particularity in the appended claims. However, a more complete understanding of the present invention may be derived by referring to the detailed description when considered in connection with the figures, wherein like reference numbers refer to similar items throughout the figures, and:

FIG. 1 is a schematic diagram of an apparatus having an internal combustion engine having a fuel tank and combustion chamber for a mixture of fuel and insecticide, according to an example embodiment.

FIG. 2 is a schematic diagram of an apparatus having an internal combustion engine having a fuel tank, an insecticide tank and combustion chamber for a mixture of fuel and insecticide, according to an example embodiment.

FIG. 3 is a schematic representation of a fuel additive, according to an example embodiment.

FIG. 4 is a process flow diagram for a method of distributing an insecticide, according to an example embodiment.

FIG. 5 is a schematic diagram of an apparatus having an internal combustion engine with an exhaust system and having an insecticide reservoir, according to an example embodiment.

FIG. 6 is a process flow diagram for a method of distributing an insecticide, according to another example embodiment.

The description set out herein illustrates the various embodiments of the invention, and such description is not intended to be construed as limiting in any manner.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

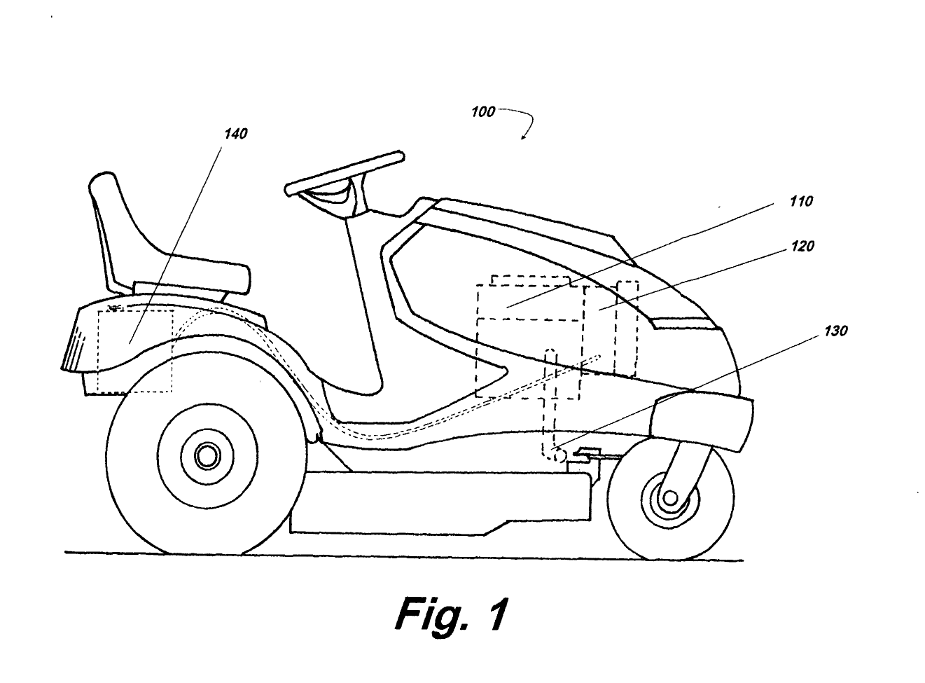

FIG. 1 is a schematic diagram of an apparatus 100 having an internal combustion engine 110 and a combustion chamber 120 for combusting a mixture of fuel and insecticide, according to an example embodiment. The apparatus 100 distributes the insecticide. The internal combustion engine 110 includes the combustion chamber 120 for burning a mixture of fuel and insecticide, and an exhaust port 130 for placing the combusted fuel and combusted insecticide into the atmosphere near the internal combustion engine 110. Uncombusted insecticide is placed in the fuel and delivered to the combustion chamber 120 where the combination of fuel and insecticide powers at least a portion of the apparatus 100. The apparatus can be any type of apparatus that includes an internal combustion engine, such as a riding lawnmower, push lawnmower, tractor, bush whacker, weed whacker, stump grinder, wood splitter, wood chipper, chainsaw, sod cutter, all terrain vehicle, motorcycle or the like. In addition, any type of internal combustion engine can be used. For example, the internal combustion engine can be a two-cycle engine, a four-cycle engine, an otto cycle type engine, a diesel cycle engine, or any other type of internal combustion engine. In the combustion chamber 120, the fuel and insecticide combust. After combustion, the byproducts of combustion leave the combustion chamber 120 via an exhaust port 130. One of the byproducts of combustion is the combusted insecticide which is delivered to the atmosphere near the apparatus 100. The apparatus 100 also includes a fuel tank 140. In the embodiment shown, the fuel tank 140 holds a mixture of fuel and an insecticide. The insecticide may be added to the fuel tank directly or may be added to the fuel tank 140 as a fuel additive.

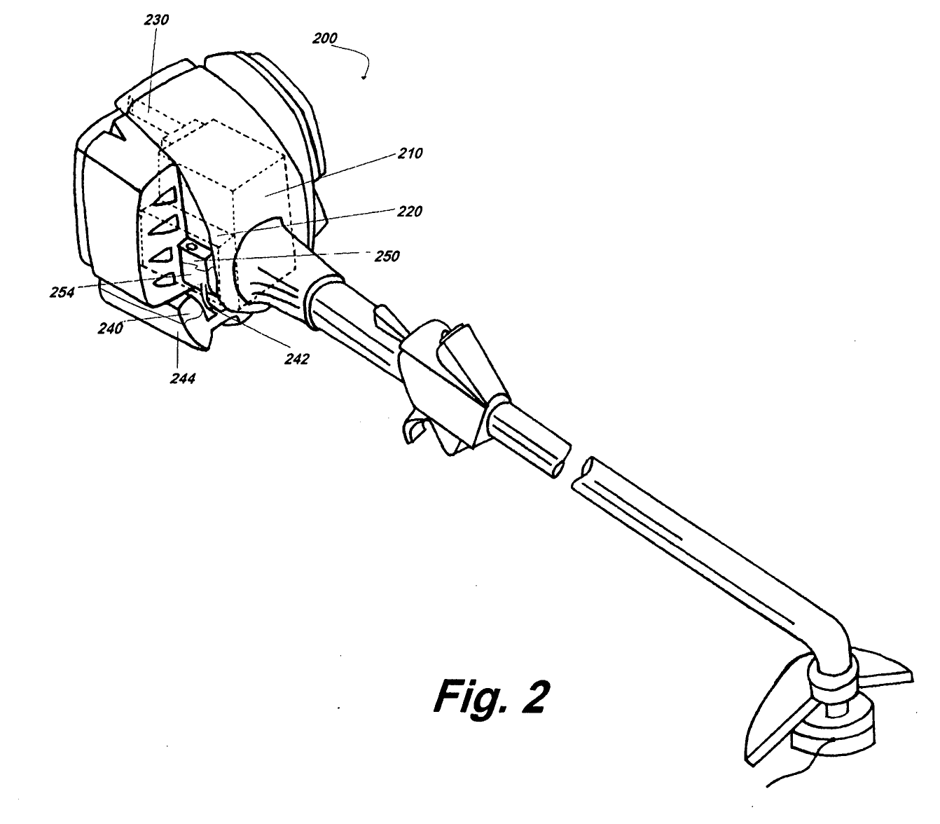

FIG. 2 is a schematic diagram of an apparatus 200 having an internal combustion engine 210 having a fuel tank 240 for holding fuel 244, an insecticide tank or reservoir 250 for holding an insecticide 254, and combustion chamber 220 for combusting a mixture of fuel 244 and insecticide 254, according to an example embodiment. In this example embodiment, the insecticide reservoir 250 has an input 252 to a fuel line 242. The insecticide reservoir 250 and the input 252 are disposed to place an insecticide 254 into the fuel 244 for input to the combustion chamber 220. As the internal combustion engine runs, the mixture of fuel 244 and insecticide 254 is combusted in the combustion chamber 220. The byproducts are exhausted or removed from the internal combustion engine 210 at the exhaust port 230. The exhaust port 230 includes the valves which open at the appropriate time to allow the products of combustion to exit the combustion chamber through an exhaust pipe.

It should be noted that the insecticide 254 for use in an internal combustion engine, such as internal combustion engine 110, 210 should not be used in concentrations that will foul the internal combustion engine. Furthermore, the insecticide selected should not include waxes or paraffin which may foul the engine. The byproducts of the combusted insecticide should combust to produce the insect repellant and not produce byproducts which will harm or drastically shorten the life of the internal combustion engine. One type of insecticide that can be combusted in an internal combustion engine includes any of various synthetic compounds that are related to the pyrethrins and resemble them in insecticidal properties. Generally, any of these synthetic compounds are referred to as a pyrethroid. Examples of the suitable pyrethroid insecticide that can be used in this invention include (+)-3-allyl-2-methyl-4-oxo-2-cyclopentenyl-(+)-trans-chrysanthemate (also generally referred to as “d-transallethrin”), (.+−.)-3-allyl-2-methyl-4-oxo-2-cyclopentenyl-(.+−.)-cis/trans-chrysanthemate (hereunder generally referred to as “allethrin”), 5-benzyl-3-furylmethyl-(.+−.)-cis/trans-chrysanthemate (hereunder generally referred to as “resmethrin”), 5-benzyl-3-furylmethyl-(+)-cis/trans-chrysanthemate (hereunder referred to as “d-resmethrin”), N-(3,4,5,6-tetrahydrophthalimido)methyl-(.+−.)-cis/trans-chrysanthemate (hereunder generally referred to as “phthalthrin”), and 3-phenoxybenzyl-(+)-cis/trans-chrysanthemate (hereunder generally referred to as “d-phenothrin”). Another specific insecticide used in one example embodiment is D-CIS|transallethrin. In another embodiment an insecticide, such as allethrin is combined with piperonyl butoxide as a volatilization controlling agent and at least one member selected from the group consisting of 2,5-di-t-butylhydroquinone. 3,5-di-t-hydroxytoluene and 3-t-butyl-4-hydroxyanisole as a volatilization controlling agent.

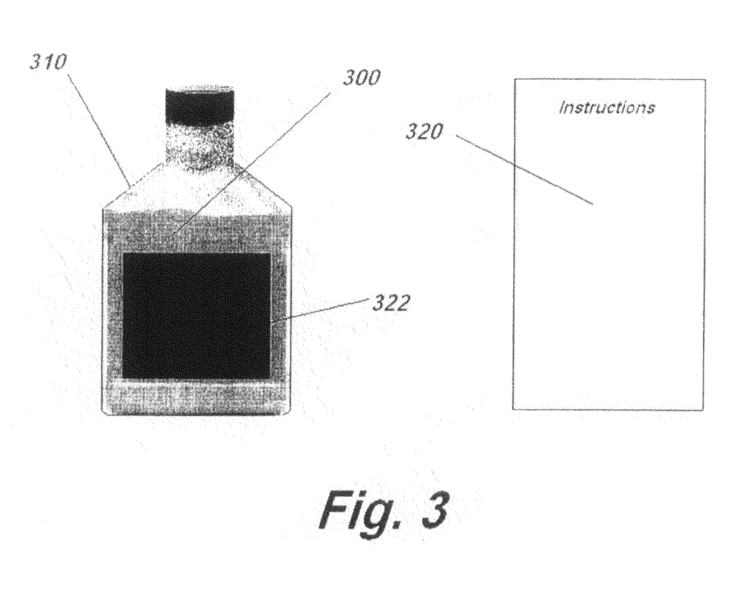

FIG. 3 is a schematic representation of a fuel additive 300, according to an example embodiment. As mentioned above, the insecticide can be directly input to the fuel of an internal combustion engine or may be added to the fuel as a fuel additive 300. The fuel additive 300 includes a base substance miscible with a fuel 244, and an insecticide 254 miscible with the fuel 244 (see FIG. 2) and the base substance. The base substance can be an oil, or can be ethanol, or any other substance that can be combusted within an internal combustion engine 110, 210 without causing excessive fouling or the like. The insecticide of the fuel additive can be any type of an insecticide, including a pyrethroid. In some embodiments, the insecticide is D-CIS|transallethrin. The fuel additive 300 can come in a kit form with a container 310 and an instruction set 320. In some embodiments, the instruction set 320 is separate from the container 310. In other embodiments, an instruction set 322 is provided on the container 310. In still another embodiment, the instruction set can be both on the container 310 and separate from the container 310. The fuel additive 300 includes insecticide is in the range of 10 to 30% of the fuel additive 300. In still another embodiment, the insecticide is in the range of 18 to 24% of the fuel additive 300. In one example embodiment, the insecticide includes a compound including a pyrethroid and another insecticide. It should be noted that the fuel additive is not limited to a pyrethroid insecticide or to any other type of insecticide. The insecticide may also include a mixture of two or more insecticides. Furthermore, the instruction set 320, 322 generally includes various warnings as well as directions regarding the use of the fuel additive. The instruction set 320, 322 generally includes specific instructions as to how much of the fuel additive to add to a unit of fuel (liters or gallons).

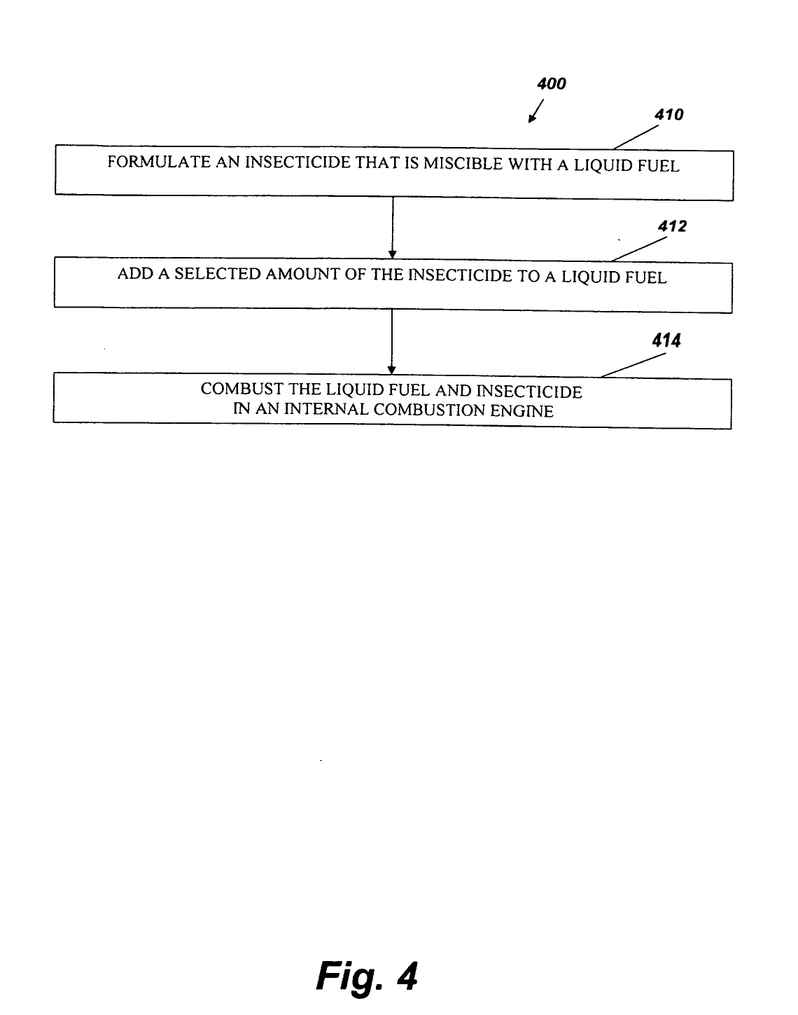

FIG. 4 is a process flow diagram for a method of distributing an insecticide, according to an example embodiment. The method 400 includes formulating an insecticide that is miscible with a liquid fuel 410, adding a selected amount of the insecticide to a liquid fuel 412, and combusting the liquid fuel and insecticide in an internal combustion engine 414. The method 400 can be used on a variety of internal combustion engines such as two-cycle engines, four-cycle engines, and engines using either an otto cycle or a diesel cycle. In some embodiments, the insecticide is mixed with the liquid fuel 412 in a ratio in the range of 4 to 1 to 2000 to 1. In other embodiments, the insecticide is mixed with the liquid fuel 412 in a ratio in the range of 10 to 1 to 500 to 1. In still other embodiments, the insecticide is mixed with the liquid fuel 412 in a ratio in the range of 30 to 1 to 100 to 1. Adding a selected amount of the insecticide to a liquid fuel 412 includes adding pyrethroid to the fuel. In some embodiments, adding a selected amount of the insecticide to a liquid fuel 412 includes adding D-CIS|transallethrin to the fuel. The insecticide can be added to the fuel as a fuel additive or can be added straight into the fuel.

There are many advantages associated with the method 400. One advantage is that the insecticide is delivered in the area of the user of an apparatus, such as apparatus 100 or 200. In other words, the insecticide is delivered as exhaust from the internal combustion engine. The exhaust is generally in the vicinity of the internal combustion engine while it is being used.

### EXAMPLE EMBODIMENT

1. Obtain D-CIS|transallethrin from a supplier such as Hangzhou Jiacheng Chemical Co., Ltd. of Hangzhou, China.
2. Mix a portion of D-CIS|transallethrin with oil in a selected proportion.
3. Bottle mixture in units for consumers

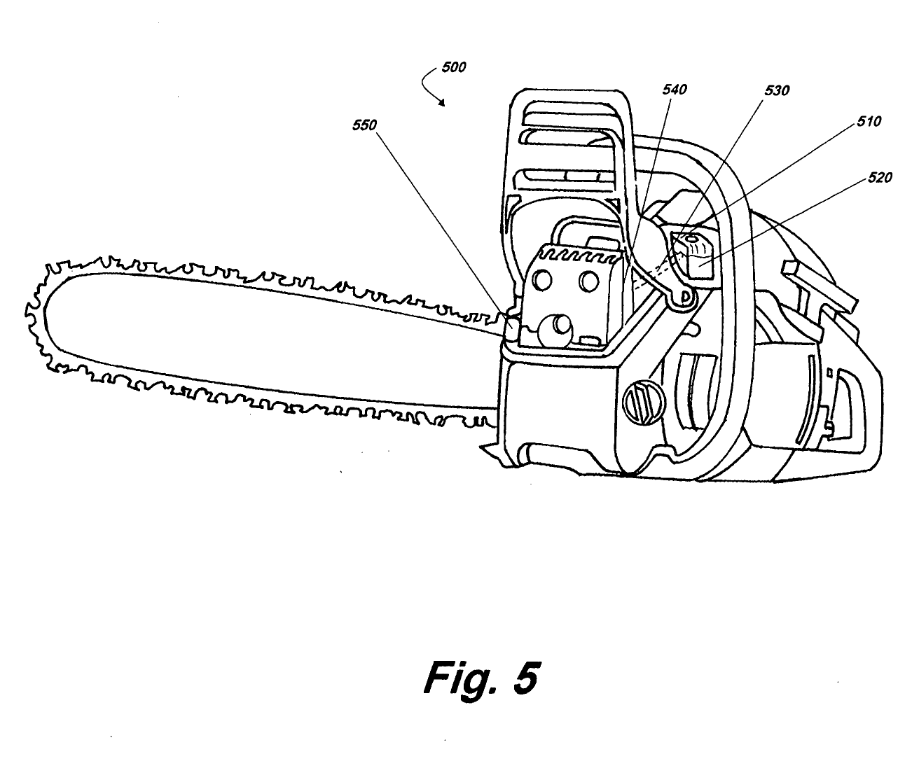

FIG. 5 is a schematic diagram of an apparatus 500 having an internal combustion engine with an exhaust system 540, and having an insecticide reservoir 510, according to an example embodiment. The insecticide reservoir 510 includes insecticide 520 is also coupled to the exhaust system 540, as depicted by a fluid path 530 from the insecticide reservoir 520 and the exhaust system 510. The fluid path 530 includes an inlet to the exhaust system 540. The inlet to the exhaust system 540 can be positioned to place an insecticide from the insecticide reservoir 510 onto a hot surface heated by the exhaust gases associated with the exhaust system 540. For example, the inlet can be positioned to place the insecticide onto a surface inside or outside of a muffler associated with the exhaust system 540. In another embodiment, the inlet can be positioned to place the insecticide onto an exhaust manifold. In the alternative, the inlet 530 to the exhaust system can be positioned to place an insecticide from the insecticide reservoir 520 into a hot stream of exhaust gases. The insecticide 520 is heated and vaporized or combusted and passed through the exhaust system 540 and out an exhaust port 550.

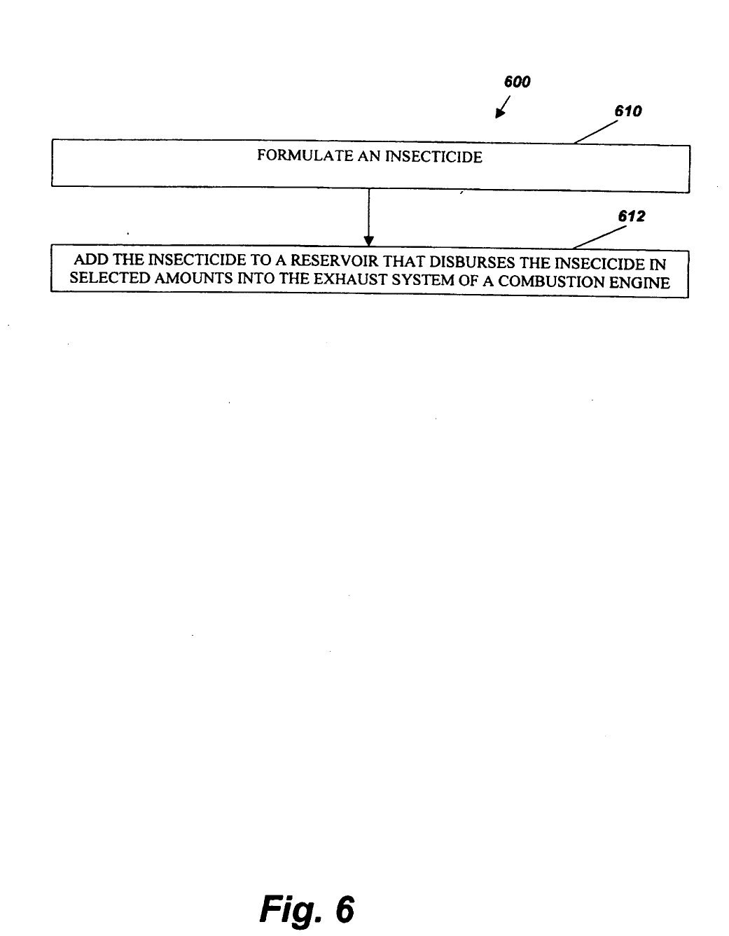

FIG. 6 is a process flow diagram for a method 600 of distributing an insecticide, according to another example embodiment. The method 600 includes formulating an insecticide 610. Formulating the insecticide 600 includes obtaining an insecticide, obtaining several insecticides and combining them, obtaining an insecticide and mixing it with another base material such as oil, or the like. The formulated insecticide 520 is placed in an insecticide reservoir 510. The insecticide reservoir 510 has a fluid path 530 that includes an inlet to the exhaust system 540. The formulated insecticide 520 is then placed on or in the exhaust system 540. The insecticide or formulated insecticide, when placed in the exhaust system 540, can be heated using the heat of the exhaust or may be combusted. The heated or combusted insecticide generally changes phase. The insecticide or formulated insecticide 520 is generally a liquid when in the insecticide reservoir 510 and is transformed to a gaseous state as a result of combustion or obtaining the heat of vaporization from the exhaust gases of the exhaust system 540. In either case, the insecticide or formulated insecticide 520 is in a gaseous state and is distributed with the exhaust gases from the exhaust system 540. The user is generally in the vicinity of the machine being operated. Therefore, the insecticide is generally dispersed in the area of the user or in the area near the user by the exhaust port 550. The user does not have to move a candle or fogger or other insecticide device, since the user is using, riding, or holding the machine being operated. The same is true for the method 400 described above (see FIG. 4).

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A method comprising:
formulating an insecticide that is miscible with a liquid fuel;
adding a selected amount of the insecticide to a liquid fuel; and
combusting the liquid fuel and insecticide in an internal combustion engine.

2. The method of claim 1 wherein the internal combustion engine uses an otto cycle.

3. The method of claim 1 wherein the internal combustion engine uses a diesel cycle.

4. The method of claim 1 wherein the internal combustion engine is a two-cycle engine.

5. The method of claim 1 wherein the internal combustion engine is a four-cycle engine.

6. The method of claim 1 wherein the insecticide is mixed with the liquid fuel in a ratio in the range of 4 to 1 to 2000 to 1.

7. The method of claim 1 wherein the insecticide is mixed with the liquid fuel in a ratio in the range of 10 to 1 to 500 to 1.

8. The method of claim 1 wherein the insecticide is mixed with the liquid fuel in a ratio in the range of 30 to 1 to 100 to 1.

9. The method of claim 1 wherein adding a selected amount of the insecticide to a liquid fuel includes adding pyrethroid to the fuel.

10. The method of claim 1 wherein adding a selected amount of the insecticide to a liquid fuel includes adding D-CIS|transallethrin to the fuel.

11. A fuel additive comprising:
a base substance miscible with a fuel; and
an insecticide miscible with the fuel and the base substance.

12. The fuel additive of claim 11 wherein the base substance is an oil.

13. The fuel additive of claim 11 wherein the base substance is ethanol.

14. The fuel additive of claim 11 wherein the insecticide is a pyrethroid.

15. The fuel additive of claim 11 wherein the insecticide is D-CIS|transallethrin.

16. The fuel additive of claim 11 wherein the insecticide includes D-CIS|transallethrin.

17. The fuel additive of claim 11 further including a container.

18. The fuel additive of claim 17 further including an instruction set.

19. The fuel additive of claim 11 wherein the insecticide is in the range of 10 to 30% of the fuel additive.

20. The fuel additive of claim 11 wherein the insecticide is in the range of 18 to 24% of the fuel additive.

21. The fuel additive of claim 11 wherein the insecticide includes a compound including a pyrethroid.

22. An apparatus for distributing an insecticide comprising:
an internal combustion engine further including:
a combustion chamber for burning a mixture of fuel and insecticide; and
an exhaust port for placing the combusted fuel and insecticide into the atmosphere near the internal combustion engine.

23. The apparatus of claim 22 further including a fuel tank for holding a mixture of fuel and an insecticide.

24. The apparatus of claim 22 further comprising:
a fuel tank;
a insecticide reservoir having an input to the fuel, the insecticide reservoir disposed to place insecticide into the fuel for input to the combustion chamber.

25. An apparatus for distributing an insecticide comprising:
an internal combustion engine further including:
a combustion chamber for burning fuel; and
an exhaust system; and
an insecticide reservoir; and
a device for coupling the insecticide reservoir and the exhaust system, the device for coupling the insecticide reservoir including an inlet to the exhaust system.

26. The apparatus of claim 25 wherein the inlet is placed to place insecticide into a stream of exhaust gases passing through the exhaust system.

27. The apparatus of claim 25 wherein the inlet is placed to place insecticide onto a surface heated by a stream of exhaust gases passing through the exhaust system.

28. A method comprising:
formulating an insecticide; and
inputting a selected amount of the insecticide to an exhaust system of an engine.
